(19) United States
(12) Patent Application Publication
(10) Pub. No.: US 2022/0370541 A1
(43) Pub. Date: Nov. 24, 2022

(54) ACTIVATED INSULIN, COMPOUND MOMORDICA CHARANTIA PEPTIDE ORAL MEDICINE FOR TREATMENT OF DIABETES, AND PREPARATION METHOD

(71) Applicants: Yubao LI, Huanggang (CN); Jingren HE, Wuhan (CN); GUOZHONGXINGHE BIOMEDICINE TECHNOLOGY CO., LTD., Wuhan (CN); CHINA YUNHONG HOLDINGS CO., LTD., Huanggang (CN)

(72) Inventors: Yubao LI, Huanggang (CN); Jingren HE, Wuhan (CN)

(21) Appl. No.: 17/728,981

(22) Filed: Apr. 26, 2022

Related U.S. Application Data
(63) Continuation of application No. PCT/CN2020/123458, filed on Oct. 24, 2020.

(30) Foreign Application Priority Data
Oct. 25, 2019 (CN) 201911024571.8

(51) Int. Cl. A61K 38/01; A61K 9/00; A61K 36/258; A61K 36/481; A61K 36/074; A61K 36/899; A61K 36/61; A61K 36/88; A61K 36/815; A61K 36/28; A61K 36/8994; A61K 31/736; A61K 36/185; A61K 31/702; A61P 3/10; A61K 38/10

(52) U.S. Cl. CPC A61K 38/011; A61K 9/0053; A61K 36/258; A61K 36/481; A61K 36/074; A61K 36/899; A61K 36/61; A61K 36/88; A61K 36/815; A61K 36/28; A61K 36/8994; A61K 31/736; A61K 36/185; A61K 31/702; A61P 3/10; A61K 38/10; A61K 2236/17; A61K 2236/39; A61K 2236/19; A61K 2236/53

(57) ABSTRACT

A compound bitter melon peptide (BMP) oral medicine for activating insulin and treating diabetes, including 20-30 parts by weight of BMP powder, 4-6 parts by weight of Panax quinquefolius, 10-12 parts by weight of Astragalus membranaceus, 3-5 parts by weight of Ganoderma lucidum powder, 8-10 parts by weight of Dioscorea opposita powder, 10-15 parts by weight of wheat bran, 10-12 parts by weight of Psidium guajava leaf powder, 5-10 parts by weight of onion extract, 5-10 parts by weight of Lycium barbarum, 12-15 parts by weight of Gynura procumbens extract, 1-2 parts by weight of coix seed, 5-8 parts by weight of konjac glucomannan, 8-10 parts by weight of lotus leaf and 5-8 parts by weight of xylo-oligosaccharide. A method for preparing the compound bitter melon peptide (BMP) oral medicine is also provided.

### CROSS-REFERENCE TO RELATED APPLICATIONS

This application is a continuation of international patent application No. PCT/CN2020/123458, filed on Oct. 24, which claims the benefit of priority from Chinese Patent Application No. 201911024571.8, filed on Oct. 25, 2019. The content of the aforementioned application, including any intervening amendments thereto, is incorporated herein by reference in its entirety.

### TECHNICAL FIELD

This application relates to drug research and development and biological fermentation, and more particularly to a compound bitter melon peptide (BMP) oral medicine for activating insulin and treating diabetes and a preparation thereof.

### BACKGROUND

The incidence of diabetes in the world is rising year after year. As published by the World Health Organization (WHO), there are more than 382 cases suffering from diabetes in the world, and about 114 cases suffering from diabetes in China.

Diabetes is a metabolic disease characterized by hyperglycemia. The hyperglycemia is caused by the insulin secretion deficiency, the impaired insulin action, or both, and can further result in chronic damage and dysfunction of various tissues, especially the eyes, kidneys, heart, blood vessels and nerves. The incidence of diabetes and hyperglycemia is associated with congenital factors and acquired factors. In China, the rising incidence of the diabetes is mainly due to the change in the dietary pattern, especially the increased proportion of pure energy food and animal fat in the daily diet.

At present, a western medicine is often taken at the early stage of diabetes to rapidly lower the blood glucose. Unfortunately, the administration of western medicines is usually accompanied by obvious adverse effects. In addition, it is required to increase the dosage and constantly adjust the medicine to ensure the treatment effect. Far from restoring the islet function and reducing the blood glucose, the above-mentioned treatment strategy has led to the long-term accumulation of the toxic and side effects in the body, resulting in more severe harm.

In recent years, extensive researches have been conducted on the preventive and therapeutic effect of the bitter melon (Momordica charantia) on diabetes. It has been reported by Khana (Khana, R., et al., CONTINUOUS AMBULATORY PERITONEAL-DIALYSIS IN DIABETICS. Kidney International, 1981. 19(2): p. 391-391) in 1981 that a 11 kDa ingredient extracted from the fruit, seeds and tissues of the bitter melon exhibits good hypoglycemic activity with respect to type I and type II diabetes after subcutaneous injection. Unfortunately, this ingredient is prone to inactivation in the intestinal tract after oral administration.

In conclusion, it is of significant importance to provide a bitter melon peptide preparation with effective regulation of the blood glucose.

### SUMMARY

In view of this, an objective of this application is to provide a compound bitter melon peptide (BMP) oral medicine for activating insulin and treating diabetes and a preparation thereof, where the preparation is performed through repeated enzymatic hydrolysis, staged heating and buffer supplementation to increase the content of BMP active ingredient. The hypoglycemic effect is further improved by combining the BMP with other natural plant ingredients.

Technical solutions of this application are described as follows.

In a first aspect, this application provides a compound bitter melon peptide (BMP) oral medicine for activating insulin and treating diabetes, comprising:

20-30 parts by weight of BMP powder;

4-6 parts by weight of Panax quinquefolius;

10-12 parts by weight of Astragalus membranaceus;

3-5 parts by weight of Ganoderma lucidum powder;

8-10 parts by weight of Dioscorea opposita powder;

10-15 parts by weight of wheat bran;

10-12 parts by weight of Psidium guajava leaf powder;

5-10 parts by weight of an onion extract;

5-10 parts by weight of Lycium barbarum;

12-15 parts by weight of a Gynura procumbens extract;

1-2 parts by weight of coix seed;

5-8 parts by weight of konjac glucomannan;

8-10 parts by weight of lotus leaf; and

5-8 parts by weight of a xylo-oligosaccharide.

In an embodiment, the BMP powder is prepared through steps of:

(S11) soaking a raw material in deionized water at 25° C. for 10-12 h, wherein the raw material is selected from the group consisting of fresh bitter melon, dried bitter melon, bitter melon seeds and a combination thereof, and a weight ratio of the raw material to the deionized water is 1:5; and taking out the raw material followed by washing with deionized water 2-3 times;

(S12) drying the raw material, followed by crushing and grinding to obtain a pulp;

(S13) mixing the pulp with a buffer solution to obtain a mixed system, and recording a volume of the mixed system as an initial volume, wherein a weight ratio of the pulp to the buffer solution is 1:(3-5); and adjusting the mixed system to pH 6.8-7 followed by temperature treatment to obtain a bitter melon extract;

wherein the temperature treatment comprises steps of:

(S131) heating the mixed system to 45-55° C. and keeping the mixed system at 45-55° C. for 45-60 min; cooling the mixed system to 20-25° C., and keeping the mixed system at 20-25° C. for 25-30 min; recording a volume of the mixed system at this time as a first volume;

(S132) introducing a first mixed solution to the mixed system, wherein the first mixed solution is composed of deionized water and the buffer solution in a weight ratio of 4:1, and a volume of the first mixed solution is 60% of a difference between the initial volume and the first volume; heating the mixed system to 60-75° C., and keeping the mixed system at 60-75° C. for 60-75 min; cooling the mixed system to 45-55° C. and keeping the mixed system at 45-55° C. for 30-35 min; recording a volume of the mixed system at this time as a second volume; and

(S133) introducing a second mixed solution to the mixed system, wherein the second mixed solution is composed of deionized water and the buffer solution in a weight ratio of 3:1, and a volume of the second mixed solution is 75% of a difference between the initial volume and the second volume; heating the mixed system to 80-90° C., and keeping the mixed system at 80-90° C. for 75-85 min; and cooling the mixed system to 60-75° C. and keeping the mixed system at 60-75° C. for 35-45 min;

(S14) cooling the bitter melon extract to 20-25° C. followed by enzymatic hydrolysis to obtain an enzymatic hydrolysis product;

wherein the enzymatic hydrolysis comprises:

adjusting the extract to pH 7.5-8.5; adding trypsin to the extract followed by heating to 35-40° C. under stirring at 80-100 rpm and keeping at 35-40° C. for 45-60 min to obtain a first enzymatic hydrolysis system, wherein the trypsin is 5% by weight of the extract;

cooling the first enzymatic hydrolysis system to 20-25° C., and adjusting the first enzymatic hydrolysis system to pH 3.0-4.0; adding pectinase to the first enzymatic hydrolysis system followed by heating to 45-55° C. under stirring at 80-100 rpm and keeping at 45-55° C. for 40-60 min to obtain a second enzymatic hydrolysis system, wherein the pectinase is 3% by weight of the first enzymatic hydrolysis system; and

cooling the second enzymatic hydrolysis system to 20-25° C. followed by adjustment to pH 4.5-5.0; and adding a cellulase to the second enzymatic hydrolysis system followed by heating to 55-60° C. under stirring at 80-100 rpm and keeping at 55-60° C. for 30-45 min to obtain the enzymatic hydrolysis product, wherein the cellulase is 2% by weight of the second enzymatic hydrolysis system;

(S15) heating the enzymatic hydrolysis product to 90° C. followed by keeping at 90° C. for 10 min for inactivation, so as to obtain a crude BMP extraction system; adding activated carbon to the crude BMP extraction system followed by uniform stirring, wherein the activated carbon is 4-5% by weight of the crude BMP extraction system; and keeping the crude BMP extraction system at 65° C. for 60-90 min followed by centrifugation to collect a first supernatant;

filtering the first supernatant with diatomite at a pressure of 0.2-0.3 MPa to obtain a first filtrate; and adding activated carbon to the first filtrate followed by standing for 45-50 min and centrifugation to collect a second supernatant, wherein the activated carbon is 4-5% by weight of the first filtrate;

(S16) filtering the second supernatant with a ceramic microfiltration membrane having a pore size of 0.5-0.8 μm at 55-65° C. to obtain a second filtrate;

filtering the second filtrate with a spiral-wound ultrafiltration membrane at 45-50° C. to obtain a third filtrate, wherein the spiral-wound ultrafiltration membrane has a molecular weight cut-off of 100-200 kDa; and

concentrating the third filtrate via a spiral-wound reverse-osmosis membrane with a molecular weight cut-off of 150-1000 Da at a temperature below 40° C. to remove water, residual inorganic salts and small molecular impurities, so as to obtain a BMP concentrate; and

(S17) subjecting the BMP concentrate to vacuum freeze drying to obtain the BMP powder, wherein the BMP powder comprises 30% or more by weight of BMP.

In an embodiment, the buffer solution is phosphate buffered saline (PBS).

In an embodiment, the Gynura procumbens extract is prepared through steps of:

(S21) selecting a Gynura procumbens plant without mildew, spots, diseases and insect pests followed by washing and drying at 100° C. in an oven for 60-90 min; and crushing a dried Gynura procumbens plant followed by sieving with a 30-50 mesh sieve to obtain Gynura procumbens powder;

(S22) mixing the Gynura procumbens powder and water in a weight ratio of 1:30 followed by stirring to obtain a mixed system, recording a weight of the mixed system as initial weight; and subjecting the mixed system to temperature treatment to obtain an enzymatic hydrolysis product;

wherein the temperature treatment comprises steps of:

(S221) heating the mixed system to 35-45° C., and keeping the mixed system at 35-45° C. for 45-60 min; cooling the mixed system to 20-25° C., and keeping the mixed system at 20-25° C. for 25-30 min; and recording a weight of the mixed system at this time as first weight;

(S222) adding a first mixed solution to the mixed system, wherein the first mixed solution is composed of deionized water and ligninase in a weight ratio of 1:0.03, and a weight of the first mixed solution is 50% of a difference between the initial weight and the first weight; heating the mixed system to 50-55° C., and keeping the mixed system at 50-55° C. for 60-75 min; cooling the mixed system to 35-55° C. and keeping the mixed system at 35-55° C. for 30-35 min; and recording a weight of the mixed system at this time as second weight;

(S223) adding a second mixed solution to the mixed system, wherein the second mixed solution is composed of deionized water and cellulase in a weight ratio of 1:0.05, and a weight of the second mixed solution is 50% of a difference between the initial weight and the second weight; heating the mixed system to 55-60° C., and keeping the mixed system at 55-60° C. for 45-60 min; cooling the mixed system to 35-40° C., and keeping the mixed system at 35-40° C. for 30-40 min, and recording a weight of the mixed system at this time as third weight; and

(S224) adding a third mixed solution to the mixed system, wherein the third mixed solution is composed of deionized water and pectinase in a weight ratio of 1:0.04, and a weight of the third mixed solution is 50% of a difference between the initial weight and the third weight; heating the mixed system to 45-50° C. and keeping the mixed system at 45-50° C. for 60-70 min; and cooling the mixed system to 20-25° C., and keeping the mixed system at 20-25° C. for 30-35 min to obtain the enzymatic hydrolysis product;

wherein a ratio of a weight of the Gynura procumbens powder to a total weight of ligninase, cellulase and pectinase is 1:0.5;

(S23) filtering the enzymatic hydrolysis product to obtain a first filtrate and a first filter residue;

(S24) subjecting the first filter residue to ultrasonic extraction in a first solution at 30-35° C. and an ultrasonic power of 100 KW for 35-45 min followed by vacuum filtration to obtain a second filtrate and a second filter residue, wherein the first solution is a 60% (v/v) ethanol solution, and a weight ratio of the first filter residue to the first solution is 1:(20-25);

subjecting the second filter residue to ultrasonic extraction in a second solution at 30-35° C. and an ultrasonic power of 100 KW for 20-30 min followed by vacuum filtration to obtain a third filtrate and a third filter residue, wherein the second solution is a 60% (v/v) ethanol solution, and a weight ratio of the second filter residue to the second solution is 1:(15-20); and

combining the first filtrate, the second filtrate and the third filtrate to obtain a Gynura procumbens crude extract; and

(S25) subjecting the Gynura procumbens crude extract to separation and purification through simulated moving bed chromatography to obtain a purified Gynura procumbens extract; concentrating the purified Gynura procumbens extract via a vacuum concentrator at 70-75° C. to obtain a Gynura procumbens concentrate; subjecting the Gynura procumbens concentrate to spray drying by using a spray dryer to obtain the Gynura procumbens extract, wherein an inlet air temperature of the spray dryer is 120-130° C. and an outlet air temperature of the spray dryer is 40-50° C.

In a second aspect, this application provides a method for preparing the compound BMP oral medicine mentioned above, comprising:

(S100) preparing the BMP powder and the Gynura procumbens extract;

(S200) weighing individual ingredients of the compound BMP oral medicine;

(S300) immersing Panax quinquefolius, Astragalus membranaceus, the Ganoderma lucidum powder, the Dioscorea opposita powder, the wheat bran, the Psidium guajava leaf powder, the onion extract, coix seed, lotus leaf and Lycium barbarum in water for 5-8 h followed by boiling for 1-2 h and filtration to obtain a first filtrate and a first filter residue, wherein a ratio of a total weight of the Panax quinquefolius, Astragalus membranaceus, Ganoderma lucidum powder, Dioscorea opposita powder, wheat bran, Psidium guajava leaf powder, onion extract, coix seed, lotus leaf and Lycium barbarum to a weight of the water is 1:(5-8);

(S400) drying the first filter residue; and soaking the first filter residue in a 70% (v/v) ethanol solution for 1-2 h followed by heating to 55-65° C., extraction for 1.5-2 h, standing at 6-9° C. for 24 h and filtration to obtain a second filtrate and a second filter residue, wherein a weight ratio of the first filter residue to the 70% (v/v) ethanol solution is 1:(2-3), and during the extraction, stirring is performed once every 10 min at 200-250 rpm;

(S500) soaking the second filter residue in a 70% (v/v) ethanol solution for 3-5 h followed by heating to 60-70° C., extraction two or three times each for 2.5-3 h, standing at 6-9° C. for 24 h and filtration to obtain a third filtrate and a third filter residue, wherein a weight ratio of the second filter residue to the 70% (v/v) ethanol solution is 1:(0.8-1); and combining the first filtrate, the second filtrate, and the third filtrate;

(S600) filtering a combined filtrate with an ultrafiltration membrane having a molecular weight cut-off of 6000-10000 Da, or with diatomite to obtain a fourth filtrate; adding the BMP powder and Gynura procumbens extract to the fourth filtrate to obtain a raw material mixture; and subjecting the raw material mixture to vacuum concentration at 50-60° C. in a vacuum concentration tank to obtain a concentrate with a relative density of 1.10-1.15 at 80° C.; and

(S700) mixing the concentrate, the konjac glucomannan and the xylo-oligosaccharide in a compounding tank under stirring followed by boiling to obtain the compound BMP oral medicine with a relative density of 1.05-1.10 at 60° C.

In an embodiment, in step (S600) further comprises: prior to adding the BMP powder and the Gynura procumbens extract, filtering the combined filtrate with diatomite.

In an embodiment, the step (S600) further comprises:

subjecting the concentrate to standing at 4° C. for 36 h or more and centrifugation at 15000-18000 rpm for 3-5 min

Compared to the prior art, this application has the following beneficial effects.

Through the combination of temperature treatment, repeated enzymatic hydrolysis and multi-filtration, the resultant BMP powder has relatively higher BMP content (no less than 20%), and through the combination of staged temperature treatment and repeated ultrasonic extraction, the production efficiency and purity of the Gynura procumbens extract are significantly improved. Further, after compounded with other ingredients, the BMP exhibit obvious biological activity, such as lowering blood glucose, blood pressure and blood lipids and losing weight, and can prevent the side effects caused by chemical drugs.

### DETAILED DESCRIPTION OF EMBODIMENTS

This application will be described in detail below with reference to the following examples.

As used herein, terms such as “have” “contain” and “include” do not exclude the presence or addition of one or more of other elements or combinations thereof.

It should be noted that unless otherwise specified, the test methods described in the following examples are conventional methods, and the reagents and materials are all commercially available.

### Example 1

Provided herein is a compound bitter melon peptide (BMP) oral medicine for activating insulin and treating diabetes, including 20 parts by weight of BMP powder, 4 parts by weight of Panax quinquefolius, 10 parts by weight of Astragalus membranaceus, 3 parts by weight of Ganoderma lucidum powder, 8 parts by weight of Dioscorea opposita powder, 10 parts by weight of wheat bran, 10 parts by weight of Psidium guajava leaf powder, 5 parts by weight of onion extract, 5 parts by weight of Lycium barbarum, 12 parts by weight of Gynura procumbens extract, 1 part by weight of coix seed, 5 parts by weight of konjac glucomannan, 8 parts by weight of lotus leaf, and 5 parts by weight of xylo-oligosaccharide.

The BMP powder is prepared as follows.

(S11) A raw material is soaked in deionized water at 25° C. for 10.5 h, where the raw material is selected from the group consisting of fresh bitter melon, dried bitter melon, bitter melon seeds and a combination thereof, and a weight ratio of the raw material to the deionized water is 1:5; and taking out the raw material followed by washing with deionized water 2-3 times to remove pesticide residues and impurities.

(S12) The raw material is air dried, and then crushed and ground to obtain a BMP pulp.

(S13) The pulp is mixed with a buffer solution ((the buffer solution is a buffer system containing reagents such as acid, alkali, salt, etc., for example phosphate buffered saline (PBS))) to obtain a mixed system. A volume of the mixed system is recorded as an initial volume, where a weight ratio of the pulp to the buffer solution is 1:4. The mixed system is adjusted to pH 6.8-7 followed by temperature treatment to obtain a bitter melon extract.

The temperature treatment is performed as follows.

(S131) The mixed system is heated to 50° C. and kept at 50° C. for 55 min, and then the mixed system is cooled to 22° C., and kept at 22° C. for 28 min. A volume of the mixed system at this time is recorded as a first volume.

(S132) Considering that water, acid and etc. are evaporated in step (S131), the changes may affect a solubility of acid, alkali and inorganic ions, thereby affecting the extracting effect. In this case, a first mixed solution is introduced to the mixed system, where the first mixed solution is composed of deionized water and the buffer solution in a weight ratio of 4:1, and a volume of the first mixed solution is 60% of a difference between the initial volume and the first volume. The mixed system is heated to 65° C., and kept at 65° C. for 65 min, and then cooled to 50° C. and kept at 50° C. for 32 min. A volume of the mixed system at this time is recorded as a second volume.

(S133) A second mixed solution is introduced to the mixed system, where the second mixed solution is composed of deionized water and the buffer solution in a weight ratio of 3:1, and a volume of the second mixed solution is 75% of a difference between the initial volume and the second volume. A temperature in step (S133) is higher than the temperature in step (S131), which leads to a more obvious evaporation of water, acid and etc., and thus a proportion of the second mixed solution should be increased (increased to 75%), and in the second mixed solution, the proportion of buffer solution has increased. After added with the second mixed solution, the mixed system is heated to 85° C., and kept at 85° C. for 80 min, and then cooled to 70° C. and kept at 70° C. for 40 min.

In the step (S13), by means of the staged temperature treatment, the cellular structure (such as cell wall) of individual ingredients is repeatedly impacted and destroyed under different temperature conditions. At the same time, after each temperature treatment stage, an appropriate amount of a mixture of water and buffer solution is added to compensate for the loss of water and acid, allowing the mixed system to be always in an optimal extracting environment to reach the optimal extraction effect.

(S14) The bitter melon extract is cooled to 20-25° C. followed by enzymatic hydrolysis to obtain an enzymatic hydrolysis product.

The enzymatic hydrolysis is performed as follows.

Primary Enzymatic Hydrolysis The extract is adjusted to pH 7.0. Trypsin is added to the extract followed by heating to 37° C. under stirring at 80-100 rpm and keeping at 37° C. for 55 min to obtain a first enzymatic hydrolysis system, where the trypsin is 5% by weight of the extract.

Secondary Enzymatic Hydrolysis

The first enzymatic hydrolysis system is cooled to 20-25° C., and the first enzymatic hydrolysis system is adjusted to pH 3.5. Pectinase is added to the first enzymolysis system followed by heating to 50° C. under stirring at 80-100 rpm and keeping at 50° C. for 50 min to obtain a second enzymatic hydrolysis system, where the pectinase is 3% by weight of the first enzymatic hydrolysis system;

Tertiary Enzymatic Hydrolysis

The second enzymatic hydrolysis system is cooled to 20-25° C. followed by adjustment to pH 4.7. A cellulase is added to the second enzymatic hydrolysis system followed by heating to 58° C. under stirring at 80-100 rpm and keeping at 58° C. for 35 min to obtain the enzymatic hydrolysis product, wherein the cellulase is 2% by weight of the second enzymatic hydrolysis system.

Considering that most of the ingredients are plant-derived, and contain cell walls, at different stages in step (S14), different enzymes and enzymatic hydrolysis conditions are employed to fully degrade the cell walls, such that the cellulose and pectin in the cell walls are released and fully degraded, allowing the effective ingredients (such as BMP) to be fully released to improve the extraction efficiency.

(S15) The enzymatic hydrolysis product is heated to 90° C. followed by keeping at 90° C. for 10 min for inactivation, so as to obtain a crude BMP extraction system. Activated carbon is added to the crude BMP extraction system followed by uniform stirring, where the activated carbon is 4-5% by weight of the crude BMP extraction system. The crude BMP extraction system is kept at 65° C. for 75 min followed by centrifugation to collect a first supernatant.

The first supernatant is filtered with diatomite at a pressure of 0.25 MPa to obtain a first filtrate. Activated carbon is added to the first filtrate followed by standing for 45-50 min and centrifugation to collect a second supernatant, where the activated carbon is 4-5% by weight of the first filtrate.

After the adsorption treatment of the activated carbon and diatomite, impurities such as pigments, suspended particles and colloids in a BMP enzymolysis solution are reduced, purifying the final product.

(S16) The second supernatant is filtered with a ceramic microfiltration membrane having a pore size of 0.5-0.8 μm at 60° C. to obtain a second filtrate. In this example, the microfiltration ceramic membrane adopts three membranes in parallel.

The second filtrate is filtered with a spiral-wound ultrafiltration membrane at 55-65° C. to obtain a third filtrate, where the spiral-wound ultrafiltration membrane has a molecular weight cut-off of 100-200 kDa. In this example, the spiral-wound ultrafiltration membrane is the spiral-wound ultrafiltration membrane with a molecular weight cut-off of 100-200 kDa, and the spiral-wound ultrafiltration membrane adopts two membranes in parallel.

The third filtrate is concentrated via a spiral-wound reverse-osmosis membrane with a molecular weight cut-off of 150-1000 Da at a temperature below 40° C. to remove water, residual inorganic salts and small molecular impurities, so as to obtain a BMP concentrate. In this example, a solid content in the BMP concentrate is no less than 40%, where the spiral-wound reverse-osmosis membrane system is a high-pressure concentrated membrane, which is made of composites membranes such as polysulfone (PS) membrane or polyethersulfone (PFS) membrane. And the spiral-wound ultrafiltration membrane adopts four membranes in series.

In step (S16), the BMP protein is separated and purified via a multi-layer membrane separation and purification technology at a low concentration temperature, so as to ensure the natural activity and high content of the BMP.

(S17) The BMP concentrate is subjected to vacuum freeze drying to obtain the BMP powder, where the BMP powder contains 30% or more by weight of BMP.

In addition, Gynura procumbens contains multiple active ingredients, such as alkaloids, coumarins, flavonoids, benzofurans, polysaccharides, organic acids, which have distinguished effect of lowering the blood glucose and blood lipids. As a result, the Gynura procumbens extract is prepared as follows.

(S21) A Gynura procumbens plant without mildew, spots, diseases and insect pests is selected followed by washing and drying at 100° C. in an oven for 75 min. A dried Gynura procumbens plant is crushed followed by sieving with a 30-50 mesh sieve to obtain Gynura procumbens powder.

(S22) The Gynura procumbens powder and water in a weight ratio of 1:30 are added followed by stirring to obtain a mixed system. A weight of the mixed system is recorded as initial weight. The mixed system is subjected to temperature treatment to obtain an enzymatic hydrolysis product.

The temperature treatment is performed as follows.

(S221) The mixed system is heated to 40° C., and kept at 40° C. for 50 min, and then the mixed system is cooled to 20-25° C., and kept at 20-25° C. for 25-30 min. A weight of the mixed system is recorded at this time as first weight.

(S222) A first mixed solution is added to the mixed system, where the first mixed solution is composed of deionized water and ligninase in a weight ratio of 1:0.03. A weight of the first mixed solution is 50% of a difference between the initial weight and the first weight. The mixed system is heated to 52° C., and kept at 52° C. for 65 min. After that, the mixed system is cooled to 45° C., and kept at 45° C. for 30-35 min. A weight of the mixed system at this time is recorded as second weight.

(S223) A second mixed solution is added to the mixed system, where the second mixed solution is composed of deionized water and cellulase in a weight ratio of 1:0.05, and a weight of the second mixed solution is 50% of a difference between the initial weight and the second weight. The mixed system is heated to 55-60° C., and kept at 55-60° C. for 50 min, and then cooled to 37° C., and kept at 37° C. for 35 min. A weight of the mixed system at this time is recorded as third weight.

(S224) A third mixed solution is added to the mixed system, where the third mixed solution is composed of deionized water and pectinase in a weight ratio of 1:0.04, and a weight of the third mixed solution is 50% of a difference between the initial weight and the third weight. The mixed system is heated up to 45-50° C., and held at 45-50° C. for 60-70 min, and then cooled to 20-25° C., and kept at 20-25° C. for 30-35 min to obtain the enzymatic hydrolysis product.

A ratio of a weight of the Gynura procumbens powder to a total weight of ligninase, cellulase and pectinase is 1:0.5.

Similarly, in this step, by means of the staged temperature treatment, the cellular structure (such as cell wall) of individual ingredients is repeatedly impacted and destroyed under different temperature conditions. At the same time, after each temperature treatment stage, an appropriate amount of a mixture of water and various kinds of enzymes is added to compensate for the loss of water and acid, allowing the ingredients in the cell walls to be fully decomposed under various conditions, which facilitates fully releasing the active ingredients in the cell wall of Gynura procumbens.

(S23) The enzymatic hydrolysis product is filtered to obtain a first filtrate and a first filter residue.

(S24) The first filter residue is subjected to ultrasonic extraction in a first solution at 32° C. and an ultrasonic power of 100 KW for 35-45 min followed by vacuum filtration to obtain a second filtrate and a second filter residue, where the first solution is a 60% (v/v) ethanol solution, and a weight ratio of the first filter residue to the first solution is 1:(20-25).

The second filter residue is subjected to ultrasonic extraction in a second solution at 30-35° C. and an ultrasonic power of 100 KW for 25 min followed by vacuum filtration to obtain a third filtrate and a third filter residue, where the second solution is a 60% (v/v) ethanol solution, and a weight ratio of the second filter residue to the second solution is 1:18.

The first filtrate, the second filtrate and the third filtrate are combined to obtain a Gynura procumbens crude extract.

(S25) The Gynura procumbens crude extract is subjected to separation and purification through simulated moving bed chromatography to obtain a purified Gynura procumbens extract, where an adsorbent used in the simulated moving bed chromatography includes 5-8 parts by weight of macroporous adsorption resin, 15-20 parts by weight of ethanol solution with a volume fraction of 60% as desorbent and 5-10 parts by weight of 95% (v/v) ethanol solution as resin regeneration solvent. The separation and purification through simulated moving bed chromatography is performed at an elution rate of 1-2 BV/h, a temperature of 50-60° C. and a pressure of 0.5-0.6 MPa.

The purified Gynura procumbens extract is concentrated via a vacuum concentrator at 70-75° C. to obtain a Gynura procumbens concentrate. The Gynura procumbens concentrate is subjected to spray drying by using a spray dryer to obtain the Gynura procumbens extract, where an inlet air temperature of the spray dryer is 125° C. and an outlet air temperature of the spray dryer is 45° C.

In conclusion, a purification and concentration of the Gynura procumbens extract is significantly increased by repeatedly extracting the filter residue and a combination with simulated moving bed chromatographic separation, allowing the Gynura procumbens extract to effectively reduce the blood glucose.

### Example 2

Example 2 is basically the same as Example 1, except that in Example 2, the compound BMP oral medicine includes 30 parts by weight of the BMP powder, 6 parts by weight of Panax quinquefolius, 12 parts by weight of Astragalus membranaceus, 5 parts by weight of the Ganoderma lucidum powder, 10 parts by weight of the Dioscorea opposita powder, 15 parts by weight of wheat bran, 12 parts by weight of the Psidium guajava leaf powder, 10 parts by weight of the onion extract, 10 parts by weight of Lycium barbarum, 15 parts by weight of the Gynura procumbens extract, 2 parts by weight of coix seed, 8 parts by weight of konjac glucomannan, 10 parts by weight of lotus leaf and 8 parts by weight of the xylo-oligosaccharide.

### Example 3

Example 3 is basically the same as Example 1, except that in Example 3, a compound BMP oral medicine for activating insulin and treating diabetes, including 25 parts by weight of the BMP powder, 5 parts by weight of Panax quinquefolius, 11 parts by weight of Astragalus membranaceus, 4 parts by weight of the Ganoderma lucidum powder, 9 parts by weight of the Dioscorea opposita powder, 12 parts by weight of wheat bran, 11 parts by weight of the Psidium guajava leaf powder, 7 parts by weight of the onion extract, 8 parts by weight of Lycium barbarum, 13 parts by weight of the Gynura procumbens extract, 1.5 parts by weight of coix seed, 7 parts by weight of konjac glucomannan, 9 parts by weight of lotus leaf and 7 parts by weight of the xylo-oligosaccharide.

Determination of Molecular Weight of BMP

A BMP sample extracted by the method described in Example 1 of the Chinese patent application No. 201710832199.8, titled “A novel method for producing BMP extract at low temperature through whole process, BMP extract and use thereof” is taken as Comparative Example 1. The BMP of Comparative Example 1 and the BMPs prepared in Examples 1-3 are analyzed by high-performance gel filtration chromatography method, so as to determine the molecular weight and the molecular weight distribution range. The results are exhibited in Table 1.

TABLE 1
Molecular weight and molecular weight distribution range of BMP

| | Peak area percentage | | | | Number-average molecular | | | | Weight-average molecular | | | |
|---|---|---|---|---|---|---|---|---|---|---|---|---|
| | (%, λ = 220 nm) | | | | weight | | | | weight | | | |
| | Compar- | | | | Compar- | | | | Compar- | | | |
| Molecular | ative | | | | ative | | | | ative | | | |
| weight | Exam- | Exam- | Exam- | Exam- | Exam- | Exam- | Exam- | Exam- | Exam- | Exam- | Exam- | Exam- |
| (Da) | ple 1 | ple 1 | ple 2 | ple 3 | ple 1 | ple 1 | ple 2 | ple 3 | ple 1 | ple 1 | ple 2 | ple 3 |
| >7000 | 16.35 | 9.45 | 9.79 | 9.86 | 9623 | 8131 | 8268 | 8311 | 9799 | 8199 | 8615 | 8562 |
| 7000-5000 | 22.66 | 32.64 | 33.52 | 33.69 | 6721 | 6215 | 6452 | 6141 | 6878 | 6477 | 6547 | 6458 |
| 5000-3000 | 14.32 | 19.31 | 19.99 | 20.58 | 4520 | 4202 | 4321 | 4158 | 4625 | 4325 | 4475 | 4341 |
| 3000-1000 | 21.47 | 25.91 | 23.43 | 25.01 | 1652 | 2852 | 2745 | 2902 | 2978 | 2752 | 2872 | 2958 |
| <1000 | 25.20 | 12.69 | 13.27 | 10.86 | 879 | 756 | 744 | 698 | 903 | 812 | 805 | 789 |

It can be exhibited that, in this disclosure, the BMP powder is prepared through the temperature treatment stage, allowing that the cellular structure (such as cell wall) of individual ingredients is repeatedly impacted and destroyed under different temperature conditions. At the same time, after each temperature treatment stage, an appropriate amount of a mixture of water and buffer solution is added to compensate for the loss of water and acid, allowing the mixed system to be always in an optimal extracting environment to reach the optimal extraction effect.

Moreover, through the combination of staged temperature treatment, repeated enzymatic hydrolysis and multi-level membrane separation and purification, the resultant BMP extract has 30% or more of BMP, and is free from polypeptides from other plant materials (such as soybean polypeptide). As shown in Table 1, the fragments with a molecular weight ranging from 5000 to 7000 Da account for nearly 30% of the BMP powder prepared herein, and these polypeptide fragments are the closest to the insulin in molecular weight and can effectively regulate the blood glucose. Therefore, it can be concluded that the obtained BMP extracts have excellent effect on regulating blood glucose metabolism, especially can significantly improve the ability of insulin receptors to bind the insulin, and lower the blood glucose.

Analysis of Gynura procumbens Extract

The dried Gynura procumbens material is subjected to reflux extraction three times at 80° C. in a 95% (w/w) ethanol solution each for 4 h, where a weight ratio of the dried Gynura procumbens material to the ethanol solution is 1:5. Then, the extracting liquids are combined and filtered at a pressure of 0.5 MPa to obtain a filtrate, which is subjected to spray drying with an inlet air temperature of 120° C. and an outlet air temperature of 80° C. to obtain a powdery Gynura procumbens extract as Comparative Example 2. The Gynura procumbens extract in Comparative Example 2 and the Gynura procumbens extracts prepared in Examples 1-3 are analyzed for the content and purity of chlorogenic acid, flavonoids and polysaccharide, and the results are shown in Table 2.

TABLE 2
Content and purity of chlorogenic acid, flavonoids
and polysaccharide of Gynura procumbens extract

| | Chlorogenic acid | | Flavonoids | | Polysaccharides | |
|---|---|---|---|---|---|---|
| | (mg/g) | | (mg/g) | | (mg/g) | |
| | Content | Purity | Content | Purity | Content | Purity |
| | (mg/g) | (%) | (mg/g) | (%) | (mg/g) | (%) |
| Comparative | 1.72 ± 0.58 | 56.2 | 1.57 ± 0.85 | 61.7 | 11.43 ± 1.25 | 63.4 |
| Example 2 | | | | | | |
| Example 1 | 3.64 ± 0.48 | 79.6 | 4.23 ± 0.92 | 85.6 | 16.51 ± 2.12 | 78.4 |
| Example 2 | 3.59 ± 0.15 | 83.6 | 4.52 ± 0.18 | 83.4 | 15.21 ± 1.45 | 83.1 |
| Example 3 | 3.45 ± 0.68 | 82.7 | 4.38 ± 0.39 | 84.6 | 15.87 ± 0.85 | 82.7 |

It can be demonstrated from Table 2 that the content and purity of chlorogenic acid, flavonoids and polysaccharides in the Gynura procumbens extracts prepared in Examples 1-3 are significantly improved compared to the Gynura procumbens extract prepared in Comparative Example 2, where the contents of chlorogenic acid, flavonoids and polysaccharides respectively reach up to 3.64 mg/g, 4.52 mg/g, and 16.51 mg/g, and the purities of chlorogenic acid, flavonoids and polysaccharides respectively reach up to 83.6%, 85.6%, and 83.1%. Through the staged temperature treatment, the cell structure is allowed to be repeatedly impacted and destroyed under different temperature conditions, and at the same time, an appropriate amount of water and a corresponding enzyme are supplemented to compensate for the water loss of the reaction system and allow the full degradation of the cell wall, promoting the release of the of the intracellular active ingredients. Moreover, the resultant filter residue is subjected to repeated ultrasonic extraction to improve the yield of the active ingredients. By means of the simulated moving bed chromatography, individual active ingredients are further purified.

### Example 4

Provided herein is a method of preparing a compound BMP oral medicine for activating insulin and treating diabetes, which is performed as follows.

(S100) The BMP powder and the Gynura procumbens extract are prepared according to Examples 1-3.

(S200) Individual ingredients are weighed according to the compounding ratio of any one of Examples 1-3.

(S300) Panax quinquefolius, Astragalus membranaceus, the Ganoderma lucidum powder, the Dioscorea opposita powder, wheat bran, the Psidium guajava leaf powder, the onion extract, coix seed, lotus leaf, Lycium barbarum are immersed in water for 6 h, followed by boiling for 1-2 h and filtration to obtain a first filtrate and a first filter residue, where a weight ratio a total weight of Panax quinquefolius, Astragalus membranaceus, the Ganoderma lucidum powder, the Dioscorea opposita powder, wheat bran, the Psidium guajava leaf powder, the onion extract, coix seed, lotus leaf and Lycium barbarum to a weight of water is 1:7.

(S400) The first filtered residue is dried. After that, the first filter residue is soaked in a 70% (v/v) ethanol solution for 1-2 h followed by heating to 60° C., extraction for 1.5-2 h, standing at 6-9° C. for 24 h and filtration to obtain a second filtrate and a second filter residue, where a weight ratio of the first filter residue to the 70% (v/v) ethanol solution is 1:(2-3), and during the extraction, stirring is performed once every 10 min at 220 rpm.

(S500) The second filter residue is soaked in a 70% (v/v) ethanol solution for 3-5 h followed by heating to 60-70° C., extraction two or three times each for 2.5-3 h, standing at 6-9° C. for 24 h and filtration to obtain a third filtrate and a third filter residue, where a weight ratio of the second filter residue to the 70% (v/v) ethanol solution is 1:(0.8-1). The first filtrate, the second filtrate, and the third filtrate are combined.

(S600) A combined filtrate is filtered with an ultrafiltration membrane having a molecular weight cut-off of 6000-10000 Da, or with diatomite to obtain a fourth filtrate. The BMP powder and Gynura procumbens extract are added to the fourth filtrate to obtain a raw material mixture. The raw material mixture is subjected to vacuum concentration at 55° C. in a vacuum concentration tank to obtain a concentrate with a relative density of 1.10-1.15 at 80° C.;

(S700) The concentrate, the konjac glucomannan and the xylo-oligosaccharide are mixed in a compounding tank under stirring followed by boiling to obtain the compound BMP oral medicine with a relative density of 1.05-1.10 at 60° C.

Evaluation of Hypoglycemic Effect

A total of 120 healthy male mice were numbered, and then adaptively raised under ad libitum feeding for 2 weeks. After that, 10 male mice were selected as blank control group, and the rest were only intraperitoneally injected with 5% streptozotocin (STZ) at a dose of 200 mg/kg. Those male mice suffering a coma within 2 hours after the injection were fed with glucose water until they recovered. Two days later, the fasting blood glucose were detected, and the male mice with blood glucose more than 10 were taken as a diabetic animal model.

Subsequently, the mice in the blank control group and the diabetic model group were subjected to ad libitum feeding; the mice from the positive control group were treated with 15 mg/kg d of rosiglitazone; the mice in the high-dose group were treated with 2 g/kg d of the compound BMP oral medicine of this disclosure, and subjected to ad libitum intake of food and water; the mice of the medium-dose group were treated with 1 g/kg d of the compound BMP oral medicine, and subjected to ad libitum intake of food and water; and the mice from the low-dose group were treated with 0.5 g/kg d of the compound BMP oral medicine, and subjected to ad libitum intake of food and water. During the experiment, the mice in each group were administered by intraperitoneal injection every day for consecutive 15 days. Then the mice were allowed to undergo an 8-h fasting period to measure the fasting blood glucose level, and the results were shown in Table 3.

TABLE 3
Effect of the compound BMP oral
medicine on blood glucose in mice

| | | | Fasting blood |
|---|---|---|---|
| | | The number | glucose level |
| Group | | of mice | (mmol/L) |
| Blank control group | | 20 | 5.41 ± 0.63 |
| Diabetic animal model group | | 20 | 15.16 ± 2.28 |
| Positive control group | | 20 | 7.24 ± 0.85 |
| Low-dose | Example 1 | 20 | 8.52 ± 1.25 |
| group | Example 2 | 20 | 8.23 ± 1.32 |
| | Example 3 | 20 | 7.94 ± 1.41 |
| Medium-dose | Example 1 | 20 | 7.53 ± 1.38 |
| group | Example 2 | 20 | 7.64 ± 1.44 |
| | Example 3 | 20 | 7.42 ± 1.20 |
| High-dose | Example 1 | 20 | 5.69 ± 0.92 |
| group | Example 2 | 20 | 5.54 ± 0.87 |
| | Example 3 | 20 | 5.56 ± 0.98 |

The results in Table 3 demonstrated that the fasting blood glucose level of the diabetic animal model group was obviously higher than the fasting blood glucose level of the blank control group. Moreover, the fasting blood glucose levels of the low-dose, medium-dose and high-dose groups were significantly lower than that of the diabetic animal model group. Especially, the high-dose administration can lower the fasting blood glucose to be close to the normal level, indicating that the BMP oral medicine provided herein has superior activity of lowering the blood glucose level of diabetic mice.

Evaluation of Activity of Lowering Level of Glycosylated Hemoglobin

A total of 240 healthy male rats, weighing 180-220 g, were selected, adaptively raised for a week and randomly and averagely divided into 12 groups. 20 rats were selected as the blank control group, and the rest were given 10 mL/kg of high-fat emulsion by intragastric administration once a day for consecutive 2 months. After the last intragastric administration, the rats were subjected to 12-h fasting with ad libitum water, and then intraperitoneally injected with 30 mg/kg of a streptozotocin (STZ) solution to establish a diabetic rat model.

The blank control group was intraperitoneally injected with an equal dose of a citric acid-sodium citrate buffer solution. 72 hours later, the rats were subjected to 12-h fasting with ad libitum water, and then the blood was collected from the tail to detect the fasting blood glucose. The fasting blood glucose equal to or higher than 7.0 mmol/L demonstrated that the diabetic rat model was successfully established.

Subsequently, the rats from the blank control group were subjected to ad libitum feeding of food and water; the rats from the diabetic rat model group were subjected to ad libitum feeding of food and water; the rats from the positive control group were treated with 0.15 g/kg d of metformin; the rats from the high-dose group were treated with 3.6 g/kg d of the compound BMP oral medicine of this disclosure, and subjected to ad libitum feeding of food and water; the rats of the medium-dose group were treated with 1.8 g/kg·d of the compound BMP oral medicine, and subjected to ad libitum feeding of food and water; and the rats from the low-dose group were treated with 0.9 g/kg·d of the compound BMP oral medicine, and subjected to ad libitum feeding of food and water. During the experiment, the rats of each group were subjected to intraperitoneal administration every day for consecutive 15 days, and then the rats were allowed to undergo an 8-h fasting to measure the glycosylated hemoglobin level. The results were shown in Table 4.

TABLE 4
Influence of the compound BMP oral medicine
on glycosylated hemoglobin level in rat

| | | | Glycosylated |
|---|---|---|---|
| | | The number | hemoglobin level |
| Group | | of rats | (ng/mL) |
| Blank control group | | 20 | 27.54 ± 3.41 |
| Diabetic rat model group | | 20 | 48.69 ± 3.43 |
| Positive control group | | 20 | 30.14 ± 1.25 |
| Low-dose | Example 1 | 20 | 41.78 ± 3.56 |
| group | Example 2 | 20 | 41.30 ± 1.83 |
| | Example 3 | 20 | 42.18 ± 2.03 |
| Medium-dose | Example 1 | 20 | 32.80 ± 1.69 |
| group | Example 2 | 20 | 32.14 ± 2.77 |
| | Example3 | 20 | 32.48 ± 3.19 |
| High-dose | Example 1 | 20 | 28.65 ± 3.91 |
| group | Example 2 | 20 | 29.20 ± 0.42 |
| | Example 3 | 20 | 29.18 ± 0.34 |

The results in Table 4 demonstrated that before administration, the glycosylated hemoglobin level in the diabetic animal model group was higher than the glycosylated hemoglobin level in the blank control group, indicating that the diabetic rat model was successfully established. After administration, the glycosylated hemoglobin levels of the low-dose, medium-dose and high-dose groups were significantly lowered, and lower than that of the diabetic model group (reduced by 41.2% at most), indicating that the BMP oral medicine can significantly lower the glycosylated hemoglobin level of diabetic rats.

Evaluation of Effect on Weight Loss

According to the experimental requirements, 120 healthy male mice, weighing 18.5 g-28.5 g (an average weight of 23.8 g), were selected and divided into 6 groups, and 120 healthy male rats, weighing 185.5 g-225.3 g (an average weight of 223.7 g), were selected and divided into 6 groups.

The mice and rats were grouped as follows. For the blank control group, each mouse was fed with 4 g/day of ordinary feed under ad libitum feeding of water, and each rat was fed with 30 g/day of ordinary feed under ad libitum feeding of water; for the model group, each mouse was fed with 2 g of high-fat feed and 2 g of ordinary feed every day under ad libitum feeding of water, and each rat was fed with 15 g of high-fat feed and 15 g of ordinary feed every day under ad libitum feeding of water; for the positive control group, each mouse was fed with 2 g of high-fat feed, 1 g of ordinary feed and lovastatin under ad libitum feeding of water, and each rat was fed with 15 g of high-fat feed, 10 g of ordinary feed and lovastatin under ad libitum feeding of water, where the lovastatin was mixed in the high-fat feed at 10 mg/per 1 kg of high-fat feed; for the high-dose group, each mouse was fed with 2 g of high-fat feed and 2 g of BMP oral medicine and 1 g of ordinary feed/day under ad libitum feeding of water, and each rat was fed with 15 g of high-fat feed, 10 g of BMP oral medicine and 10 g of ordinary feed/day under ad libitum feeding of water. For the medium-dose group, each mouse was fed with 2 g of high-fat feed, 1.5 g of BMP oral medicine and 1 g of ordinary feed/day under ad libitum feeding of water, and each rat was fed with 15 g of high-fat feed, 8 g of BMP oral medicine and 10 g of ordinary feed/day under ad libitum feeding of water. For the low-dose group, each mouse was fed with 2 g of high-fat feed, 0.5 g of BMP oral medicine and 1 g of ordinary feed/day under ad libitum feeding of water, and each rat was fed with 15 g of high-fat feed, 5 g of BMP oral medicine and 10 g of ordinary feed/day under ad libitum feeding of water.

After fed for 30 days, of the mice and rats in each group were measured for the weight, and the results were shown in Table 5.

TABLE 5
Influence of compound BMP oral medicine
on weight of mice and rat

| | | The number of | | | |
|---|---|---|---|---|---|
| | | mice or rats | | Weight (g) | |
| Group | | Mice | Rats | Mice | Rats |
| Blank control group | | 10 | 10 | 29.5 ± 4.1 | 248.3 ± 2.6 |
| Model group | | 10 | 10 | 34.5 ± 3.4 | 296.7 ± 4.1 |
| Positive control group | | 10 | 10 | 33.9 ± 2.3 | 255.2 ± 3.5 |
| Low-dose | Example 1 | 10 | 10 | 32.9 ± 2.6 | 275.7 ± 4.2 |
| group | Example 2 | 10 | 10 | 31.3 ± 2.4 | 278.3 ± 5.1 |
| | Example 3 | 10 | 10 | 32.1 ± 2.4 | 276.8 ± 3.3 |
| Medium-dose | Example 1 | 10 | 10 | 31.4 ± 2.9 | 261.4 ± 4.2 |
| group | Example 2 | 10 | 10 | 30.9 ± 1.8 | 259.2 ± 3.8 |
| | Example 3 | 10 | 10 | 31.2 ± 3.3 | 262.7 ± 3.3 |
| High-dose | Example 1 | 10 | 10 | 30.8 ± 2.8 | 250.2 ± 4.2 |
| group | Example 2 | 10 | 10 | 30.2 ± 2.7 | 248.7 ± 6.1 |
| | Example 3 | 10 | 10 | 30.9 ± 2.1 | 251.4 ± 3.8 |

The results in Table 5 demonstrated that after 30 days, the weight of the model group increased, and were evidently higher than the weight of the blank control group, low-dose group, medium-dose group and high-dose group. Moreover, compared with the model group, the low-dose administration and the high-dose administration have significantly reduced the weight, indicating that the BMP oral medicine contributes to the weight loss of the diabetic mouse and the diabetic rats.

Evaluation of Effect on Binding Ability of Insulin Receptor 240 healthy rats (120 male rats and 120 female rats), weighting about 160-200 g, were selected. The rats were adaptively raised for a week under ad libitum feeding of food and water. During the adaptively raising, blood was collected to measure fasting blood glucose twice. The blood glucose level of 3.6-5.4 mmol/L was an inclusion criterion the selected rats. 20 rats were selected as blank control group, and the rest were selected for modeling using tetraoxypyrimidine (at a dose of 15 mg/100 g of weight). 7-10 days after the modeling, the fasting blood glucose level was re-measured, and the rats whose fasting blood glucose level were more than 13.8 mmol/L were selected as the model successful rats.

Subsequently, the rats in the blank control group and the diabetic model group were subjected to ad libitum feeding; the rats from the positive control group were treated with 0.15 g/kg·d of dimethylbiguanide; the rats in the high-dose group were administrated with 3.6 g/kg·d of the BMP oral medicine under ad libitum feeding of food and water; the rats in the medium-dose group were administrated with 1.8 g/kg·d of the BMP oral medicine under ad libitum feeding of food and water; the rats in the low-dose group were administrated with 0.9 g/kg·d of the BMP oral medicine under ad libitum feeding of food and water;. During the experiment, the rats of each group were continuously administered by intraperitoneal injection every day. After 15 days, the rats were fasted for 8 h to test a serum insulin content. The results are shown in

### Table 6.

TABLE 6
Influence on serum insulin content of
rats by compound BMP oral medicine

| | | |
|---|---|---|
| | The number | Serum insulin |
| Group | of rats | content (mu/L) |

| Blank control group | | | 20 | 31.4 ± 6.5 |
|---|---|---|---|---|
| Model group | | | 20 | 18.3 ± 5.0 |
| | Low-dose | Example 1 | 20 | 25.2 ± 5.5 |
| | group | Example 2 | 20 | 22.1 ± 10.1 |
| | | Example 3 | 20 | 22.3 ± 10.2 |
| | Medium-dose | Example 1 | 20 | 23.2 ± 5.5 |
| | group | Example 2 | 20 | 26.4 ± 6.8 |
| | | Example 3 | 20 | 24.6 ± 7.0 |
| | High-dose | Example 1 | 20 | 24.1 ± 10.1 |
| | group | Example 2 | 20 | 28.4 ± 4.3 |
| | | Example 3 | 20 | 27.0 ± 8.3 |

The results shown in Table 6 demonstrated that the serum insulin content of the model group was evidently lower than the serum insulin content of the blank control group, indicating that tetraoxypyrimidine caused certain damage to islet β-cells. The insulin levels in the low, medium and high-dose groups were significantly higher than the insulin level of the model group, indicating that the compound BMP oral medicine has a certain repairing effect on the damaged islet β-cells, promoting the enhancement of insulin secretion, improving the binding ability of insulin receptors and strengthening the hypoglycemic effect.

Evaluation of Activity of Lowering Blood Lipids

A total of 240 healthy female rats were numbered and adaptively raised for 2 weeks under ad libitum feeding of to water. After that, the rats were randomly divided into 12 groups with 20 rats in each group, where the groups include blank control group, model group, positive control group (simvastatin), high-dose group, medium-dose group and low-dose group. Except for the blank control group, the other groups were fed with high-fat feed. 3 weeks after modeling, the rats were subjected to intragastric administration of a solution at a corresponding concentration formulated by the compound BMP oral medicine and simvastatin at a dose of 2% of the rat weight every day. The rats in the low-dose group were subjected to 25 mg/kg bw·d of intragastric administration. The rats in the medium-dose group were subjected to 50 mg/kg bw·d of intragastric administration. The rats in the high-dose group were subjected to 100 mg/kg bw·d of intragastric administration. The rats in the positive control group were subjected to intragastric administration of 50 mg/kg bw·d of simvastatin. The rats in the blank control group were subjected to intragastric administration of distilled water. The rats were continuously fed for 10 weeks. After the last feeding, the rats are fasted under ad libitum intake of water for 10 h. And then the blood was collected from aorta to test a total cholesterol (TC) content and a triglyceride (TG) content in serum. The test results are shown in Table 7.

TABLE 7
Influence of compound BMP oral medicine on serum lipids of rats

| | | | Total | |
|---|---|---|---|---|
| | | | cholesterol | Triglyceride |
| | | The number | (TC) | (TG) |
| Group | | of rats | (mmol/L) | (mmol/L) |
| Blank control group | | 20 | 1.79 ± 0.25 | 0.34 ± 0.18 |
| Model group | | 20 | 3.41 ± 0.58 | 0.57 ± 0.18 |
| Positive control group | | 20 | 2.59 ± 0.24 | 0.32 ± 0.14 |
| Low-dose | Example 1 | 20 | 3.26 ± 0.48 | 0.34 ± 0.14 |
| group | Example 2 | 20 | 3.15 ± 0.34 | 0.31 ± 0.17 |
| | Example 3 | 20 | 3.13 ± 0.42 | 0.32 ± 0.13 |
| Medium-dose | Example 1 | 20 | 2.73 ± 0.58 | 0.26 ± 0.07 |
| group | Example 2 | 20 | 2.79 ± 0.61 | 0.27 ± 0.09 |
| | Example 3 | 20 | 2.96 ± 0.63 | 0.24 ± 0.06 |
| High-dose | Example 1 | 20 | 3.57 ± 0.47 | 0.28 ± 0.06 |
| group | Example 2 | 20 | 3.64 ± 0.49 | 0.29 ± 0.04 |
| | Example 3 | 20 | 3.71 ± 0.53 | 0.26 ± 0.07 |

The results shown in Table 7 demonstrated that the compound BMP oral medicine of high, medium and low-doses could significantly lower the total cholesterol (TC) content and the triglyceride (TG) content in serum of rat (at most, the total cholesterol (TC) content can be decreased by 58% and the triglyceride (TG) content can be decreased by 20%), indicating that the compound BMP oral medicine had the effect of reducing TG content and TC content in serum, so as to lower the blood lipids.

It should be noted that the technical solutions in the above-mentioned Examples 1-4 can be combined arbitrarily, and all the combinations shall fall within the protection scope of this disclosure if there is no contradiction.

To sum up, in the preparation of the BMP powder provided herein, the temperature treatment stage was performed to allow that the cellular structure (such as cell wall) of individual ingredients is repeatedly impacted and destroyed under different temperature conditions. At the same time, after each temperature treatment stage, an appropriate amount of a mixture of water and buffer solution is added to compensate for the loss of water and acid, allowing the mixed system to be always in an optimal extracting environment to reach the optimal extraction effect. Moreover, through the combination of staged temperature treatment, repeated enzymatic hydrolysis and multi-level membrane separation and purification, the resultant BMP extract has 30% or more of BMP, and is free from polypeptides from other plant materials (such as soybean polypeptide). As shown in Table 1, the fragments with a molecular weight ranging from 5000 to 7000 Da account for nearly 30% of the BMP powder prepared herein, and these polypeptide fragments are the closest to the insulin in molecular weight and can effectively regulate the blood glucose. Therefore, it can be concluded that the obtained BMP extracts have excellent effect on regulating blood glucose metabolism, especially can significantly improve the ability of insulin receptors to bind the insulin, and lower the blood glucose, promoting the release of the of the intracellular active ingredients. Further, the resultant filter residue is subjected to repeated ultrasonic extraction to improve the yield of the active ingredients. By means of the simulated moving bed chromatography, individual active ingredients are further purified. Further, in this case, BMP and other ingredients (such as Panax quinquefolius, Astragalus membranaceus, the Ganoderma lucidum powder, the Dioscorea opposita powder, kudzu vine root and etc.) are compounded to be used to not only significantly lower the blood glucose, blood lipids and glycosylated hemoglobin level and help lose weight, but also get rid of the side effects caused by chemical drugs.

Though the disclosure has been described in detain above, it should be understood that those skilled in the art can still make some modifications and variations to the technical solutions provided herein.

Described above are merely some examples of this disclosure, which are not intended to limit this disclosure. It should be understood by those skilled in the art that any modifications, variations and replacements made without departing from the spirit and scope of this disclosure shall fall within the scope of the disclosure defined by the appended claims.

What is claimed is:

1. A compound bitter melon peptide (BMP) oral medicine for activating insulin and treating diabetes, comprising:
20-30 parts by weight of BMP powder;
4-6 parts by weight of Panax quinquefolius;
10-12 parts by weight of Astragalus membranaceus;
3-5 parts by weight of Ganoderma lucidum powder;
8-10 parts by weight of Dioscorea opposita powder;
10-15 parts by weight of wheat bran;
10-12 parts by weight of Psidium guajava leaf powder;
5-10 parts by weight of an onion extract;
5-10 parts by weight of Lycium barbarum;
12-15 parts by weight of a Gynura procumbens extract;
1-2 parts by weight of coix seed;
5-8 parts by weight of konjac glucomannan;
8-10 parts by weight of lotus leaf; and
5-8 parts by weight of a xylo-oligosaccharide.

2. The compound BMP oral medicine of claim 1, comprising:
20 parts by weight of the BMP powder;
4 parts by weight of Panax quinquefolius;
10 parts by weight of Astragalus membranaceus;
3 parts by weight of the Ganoderma lucidum powder;
8 parts by weight of the Dioscorea opposita powder;
10 parts by weight of wheat bran;
10 parts by weight of the Psidium guajava leaf powder;
5 parts by weight of the onion extract;
5 parts by weight of Lycium barbarum;
12 parts by weight of the Gynura procumbens extract;
1 part by weight of coix seed;
5 parts by weight of konjac glucomannan;
8 parts by weight of lotus leaf; and
5 parts by weight of the xylooligosaccharide.

3. The compound BMP oral medicine of claim 1, comprising:
30 parts by weight of the BMP powder;
6 parts by weight of Panax quinquefolius;
12 parts by weight of Astragalus membranaceus;
5 parts by weight of the Ganoderma lucidum powder;
10 parts by weight of the Dioscorea opposita powder;
15 parts by weight of wheat bran;
12 parts by weight of the Psidium guajava leaf powder;
10 parts by weight of the onion extract;
10 parts by weight of Lycium barbarum;
15 parts by weight of the Gynura procumbens extract;
2 parts by weight of coix seed;
8 parts by weight of konj ac glucomannan;
10 parts by weight of lotus leaf; and
8 parts by weight of the xylo-oligosaccharide.

4. The compound BMP oral medicine of claim 1, comprising:
25 parts by weight of the BMP powder;
5 parts by weight of Panax quinquefolius;
11 parts by weight of Astragalus membranaceus;
4 parts by weight of the Ganoderma lucidum powder;
9 parts by weight of the Dioscorea opposita powder;
12 parts by weight of wheat bran;
11 parts by weight of the Psidium guajava leaf powder;
7 parts by weight of the onion extract;
8 parts by weight of Lycium barbarum;
13 parts by weight of the Gynura procumbens extract;
1.5 parts by weight of coix seed;
7 parts by weight of konjac glucomannan;
9 parts by weight of lotus leaf; and
7 parts by weight of the xylo-oligosaccharide.

5. The compound BMP oral medicine of claim 1, wherein the BMP powder is prepared through steps of:
(S11) soaking a raw material in deionized water at 25° C. for 10-12 h, wherein the raw material is selected from the group consisting of fresh bitter melon, dried bitter melon, bitter melon seeds and a combination thereof, and a weight ratio of the raw material to the deionized water is 1:5; and taking out the raw material followed by washing with deionized water 2-3 times;
(S12) drying the raw material followed by crushing and grinding to obtain a pulp;
(S13) mixing the pulp with a buffer solution to obtain a mixed system, and recording a volume of the mixed system as an initial volume, wherein a weight ratio of the pulp to the buffer solution is 1:(3-5); and adjusting the mixed system to pH 6.8-7 followed by temperature treatment to obtain a bitter melon extract;
wherein the temperature treatment comprises steps of:
(S131) heating the mixed system to 45-55° C. and keeping the mixed system at 45-55° C. for 45-60 min; cooling the mixed system to 20-25° C., and keeping the mixed system at 20-25° C. for 25-30 min; recording a volume of the mixed system at this time as a first volume;
(S132) introducing a first mixed solution to the mixed system, wherein the first mixed solution is composed of deionized water and the buffer solution in a weight ratio of 4:1, and a volume of the first mixed solution is 60% of a difference between the initial volume and the first volume; heating the mixed system to 60-75° C., and keeping the mixed system at 60-75° C. for 60-75 min; cooling the mixed system to 45-55° C. and keeping the mixed system at 45-55° C. for 30-35 min; recording a volume of the mixed system at this time as a second volume; and
(S133) introducing a second mixed solution to the mixed system, wherein the second mixed solution is composed of deionized water and the buffer solution in a weight ratio of 3:1, and a volume of the second mixed solution is 75% of a difference between the initial volume and the second volume; heating the mixed system to 80-90° C., and keeping the mixed system at 80-90° C. for 75-85 min; and cooling the mixed system to 60-75° C. and keeping the mixed system at 60-75° C. for 35-45 min;
(S14) cooling the bitter melon extract to 20-25° C. followed by enzymatic hydrolysis to obtain an enzymatic hydrolysis product;
wherein the enzymatic hydrolysis comprises steps of:
adjusting the extract to pH 7.5-8.5; adding trypsin to the extract followed by heating to 35-40° C. under stirring at 80-100 rpm and keeping at 35-40° C. for 45-60 min to obtain a first enzymatic hydrolysis system, wherein the trypsin is 5% by weight of the extract;
cooling the first enzymatic hydrolysis system to 20-25° C., and adjusting the first enzymatic hydrolysis system to pH 3.0-4.0; adding pectinase to the first enzymatic hydrolysis system followed by heating to 45-55° C. under stirring at 80-100 rpm and keeping at 45-55° C. for 40-60 min to obtain a second enzymatic hydrolysis system, wherein the pectinase is 3% by weight of the first enzymatic hydrolysis system; and
cooling the second enzymatic hydrolysis system to 20-25° C. followed by adjustment to pH 4.5-5.0; and adding a cellulase to the second enzymatic hydrolysis system followed by heating to 55-60° C. under stirring at 80-100 rpm and keeping at 55-60° C. for 30-45 min to obtain the enzymatic hydrolysis product, wherein the cellulase is 2% by weight of the second enzymatic hydrolysis system;
(S15) heating the enzymatic hydrolysis product to 90° C. followed by keeping at 90° C. for 10 min for inactivation, so as to obtain a crude BMP extraction system;
adding activated carbon to the crude BMP extraction system followed by uniform stirring, wherein the activated carbon is 4-5% by weight of the crude BMP extraction system; and keeping the crude BMP extraction system at 65° C. for 60-90 min followed by centrifugation to collect a first supernatant;
filtering the first supernatant with diatomite at a pressure of 0.2-0.3 MPa to obtain a first filtrate; and adding activated carbon to the first filtrate followed by standing for 45-50 min and centrifugation to collect a second supernatant, wherein the activated carbon is 4-5% by weight of the first filtrate;
(S16) filtering the second supernatant with a ceramic microfiltration membrane having a pore size of 0.5-0.8 μm at 55-65° C. to obtain a second filtrate;
filtering the second filtrate with a spiral-wound ultrafiltration membrane at 45-50° C. to obtain a third filtrate, wherein the spiral-wound ultrafiltration membrane has a molecular weight cut-off of 100-200 kDa; and
concentrating the third filtrate via a spiral-wound reverse-osmosis membrane with a molecular weight cut-off of 150-1000 Da at a temperature below 40° C. to remove water, residual inorganic salts and small molecular impurities, so as to obtain a BMP concentrate; and
(S17) subjecting the BMP concentrate to vacuum freeze drying to obtain the BMP powder, wherein the BMP powder comprises 30% or more by weight of BMP.

6. The compound BMP oral medicine of claim 5, wherein in step (S11), the raw material is soaked at 25° C. for 10.5 h.

7. The compound BMP oral medicine of claim 5, wherein in step (S13), the weight ratio of the pulp to the buffer solution is 1:4.

8. The compound BMP oral medicine of claim 5, wherein in step (S131), the mixed system is heated to 50° C., kept at 50° C. for 55 min, cooled to 22° C. and kept at 22° C. for 28 min;
in step (S132), after the first mixed solution is added, the mixed system is heated to 65° C., kept at 65° C. for 65 min, cooled to 50° C. and kept at 50° C. for 32 min; and
in step (S133), after the second mixed solution is added, the mixed system is heated to 85° C., kept at 85° C. for 80 min, cooled to 70° C. and kept at 70° C. for 40 min.

9. The compound BMP oral medicine of claim 5, wherein the buffer solution is phosphate buffered saline (PBS).

10. The compound BMP oral medicine of claim 1, wherein the Gynura procumbens extract is prepared through steps of:
(S21) selecting a Gynura procumbens plant without mildew, spots, diseases and insect pests followed by washing and drying at 100° C. in an oven for 60-90 min; and crushing a dried Gynura procumbens plant followed by sieving with a 30-50 mesh sieve to obtain Gynura procumbens powder;
(S22) mixing the Gynura procumbens powder and water in a weight ratio of 1:30 followed by stirring to obtain a mixed system, recording a weight of the mixed system as initial weight; and subjecting the mixed system to temperature treatment to obtain an enzymatic hydrolysis product;
wherein the temperature treatment comprises steps of:
(S221) heating the mixed system to 35-45° C., and keeping the mixed system at 35-45° C. for 45-60 min; cooling the mixed system to 20-25° C., and keeping the mixed system at 20-25° C. for 25-30 min; and recording a weight of the mixed system at this time as first weight;
(S222) adding a first mixed solution to the mixed system, wherein the first mixed solution is composed of deionized water and ligninase in a weight ratio of 1:0.03, and a weight of the first mixed solution is 50% of a difference between the initial weight and the first weight; heating the mixed system to 50-55° C., and keeping the mixed system at 50-55° C. for 60-75 min; cooling the mixed system to 35-55° C. and keeping the mixed system at 35-55° C. for 30-35 min; and recording a weight of the mixed system at this time as second weight;
(S223) adding a second mixed solution to the mixed system, wherein the second mixed solution is composed of deionized water and cellulase in a weight ratio of 1:0.05, and a weight of the second mixed solution is 50% of a difference between the initial weight and the second weight; heating the mixed system to 55-60° C., and keeping the mixed system at 55-60° C. for 45-60 min; cooling the mixed system to 35-40° C., and keeping the mixed system at 35-40° C. for 30-40 min, and recording a weight of the mixed system at this time as third weight; and
(S224) adding a third mixed solution to the mixed system, wherein the third mixed solution is composed of deionized water and pectinase in a weight ratio of 1:0.04, and a weight of the third mixed solution is 50% of a difference between the initial weight and the third weight; heating the mixed system to 45-50° C. and keeping the mixed system at 45-50° C. for 60-70 min; and cooling the mixed system to 20-25° C., and keeping the mixed system at 20-25° C. for 30-35 min to obtain the enzymatic hydrolysis product;
wherein a ratio of a weight of the Gynura procumbens powder to a total weight of ligninase, cellulase and pectinase is 1:0.5;
(S23) filtering the enzymatic hydrolysis product to obtain a first filtrate and a first filter residue;
(S24) subjecting the first filter residue to ultrasonic extraction in a first solution at 30-35° C. and an ultrasonic power of 100 KW for 35-45 min followed by vacuum filtration to obtain a second filtrate and a second filter residue, wherein the first solution is a 60% (v/v) ethanol solution, and a weight ratio of the first filter residue to the first solution is 1:(20-25);
subjecting the second filter residue to ultrasonic extraction in a second solution at 30-35° C. and an ultrasonic power of 100 KW for 20-30 min followed by vacuum filtration to obtain a third filtrate and a third filter residue, wherein the second solution is a 60% (v/v) ethanol solution, and a weight ratio of the second filter residue to the second solution is 1:(15-20); and
combining the first filtrate, the second filtrate and the third filtrate to obtain a Gynura procumbens crude extract; and
(S25) subjecting the Gynura procumbens crude extract to separation and purification through simulated moving bed chromatography to obtain a purified Gynura procumbens extract; concentrating the purified Gynura procumbens extract via a vacuum concentrator at 70-75° C. to obtain a Gynura procumbens concentrate;
subjecting the Gynura procumbens concentrate to spray drying by using a spray dryer to obtain the Gynura procumbens extract, wherein an inlet air temperature of the spray dryer is 120-130° C. and an outlet air temperature of the spray dryer is 40-50° C.

11. The compound BMP oral medicine of claim 10, wherein in step (S221), the mixed system is heated to 40° C., kept at 40° C. for 50 min, cooled to 20-25° C., and kept at 20-25° C. for 25-30 min;
in step (S222), after the first mixed solution is added, the mixed system is heated to 52° C., kept at 52° C. for 65 min, cooled to 45° C. and kept at 45° C. for 30-35 min;
in step (S223), after the second mixed solution is added, the mixed system is heated to 55-60° C., kept at 55-60° C. for 50 min, cooled to 37° C., and kept at 37° C. for 35 min; and
in step (S224), after the third mixed solution is added, the mixed system is heated to 45-50° C., kept at 45-50° C. for 60-70 min, cooled to 20-25° C., and kept at 20-25° C. for 30-35 min.

12. A method for preparing the compound BMP oral medicine of claim 1, comprising:
(S100) preparing the BMP powder and the Gynura procumbens extract;
(S200) weighing individual ingredients of the compound BMP oral medicine;
(S300) immersing Panax quinquefolius, Astragalus membranaceus, the Ganoderma lucidum powder, the Dioscorea opposita powder, the wheat bran, the Psidium guajava leaf powder, the onion extract, coix seed, lotus leaf and Lycium barbarum in water for 5-8 h followed by boiling for 1-2 h and filtration to obtain a first filtrate and a first filter residue, wherein a ratio of a total weight of the Panax quinquefolius, Astragalus membranaceus, Ganoderma lucidum powder, Dioscorea opposita powder, wheat bran, Psidium guajava leaf powder, onion extract, coix seed, lotus leaf and Lycium barbarum to a weight of the water is 1:(5-8);
(S400) drying the first filter residue; and soaking the first filter residue in a 70% (v/v) ethanol solution for 1-2 h followed by heating to 55-65° C., extraction for 1.5-2 h, standing at 6-9° C. for 24 h and filtration to obtain a second filtrate and a second filter residue, wherein a weight ratio of the first filter residue to the 70% (v/v) ethanol solution is 1:(2-3), and during the extraction, stirring is performed once every 10 min at 200-250 rpm;
(S500) soaking the second filter residue in a 70% (v/v) ethanol solution for 3-5 h followed by heating to 60-70° C., extraction once or twice each for 2.5-3 h, standing at 6-9° C. for 24 h and filtration to obtain a third filtrate and a third filter residue, wherein a weight ratio of the second filter residue to the 70% (v/v) ethanol solution is 1:(0.8-1); and combining the first filtrate, the second filtrate, and the third filtrate;
(S600) filtering a combined filtrate with an ultrafiltration membrane having a molecular weight cut-off of 6000-10000 Da, or with diatomite to obtain a fourth filtrate; adding the BMP powder and Gynura procumbens extract to the fourth filtrate to obtain a raw material mixture; and subjecting the raw material mixture to vacuum concentration at 50-60° C. in a vacuum concentration tank to obtain a concentrate with a relative density of 1.10-1.15 at 80° C.; and
(S700) mixing the concentrate, the konjac glucomannan and the xylo-oligosaccharide in a compounding tank under stirring followed by boiling to obtain the compound BMP oral medicine with a relative density of 1.05-1.10 at 60° C.

13. The method of claim 12, wherein the step (S600) further comprises:
subjecting the concentrate to standing at 4° C. for 36 h or more and centrifugation at 15000-18000 rpm for 3-5 min.

14. A method for activating insulin in a subject in need thereof, comprising:
administering to the subject 0.5-2 g/(kg·d) of the compound BMP oral medicine of claim 1.

15. A method for treating diabetes in a subject in need thereof, comprising:
administering to the subject 0.5-2 g/(kg·d) of the compound BMP oral medicine of claim 1.
